(19) United States
(12) Patent Application Publication
(10) Pub. No.: US 2026/0263208 A1
(43) Pub. Date: Sep. 10, 2026

(54) IMPLANTABLE DEVICE, IN PARTICULAR FOR PARIETAL REPAIR

(71) Applicant: COUSIN BIOTECH, WERVICQ SUD (FR)

(72) Inventors: Gilles SOLECKI, LANNOY (FR); Safa OUERGHEMMI, MONS-EN-BAROEUL (FR); Michel CAILLIBOTTE, LILLE (FR)

(21) Appl. No.: 19/168,847

(22) PCT Filed: Mar. 26, 2024
(86) PCT No.: PCT/EP2024/058149

§ 371 Date: Sep. 25, 2025

(30) Foreign Application Priority Data
Mar. 27, 2023 (FR) FR2302903

(51) Int. Cl. A61F 2/00; B32B 5/02; B32B 5/26; D04B 21/06; D04B 21/20

(52) U.S. Cl. CPC A61F 2/0063; B32B 5/026; B32B 5/26; D04B 21/06; D04B 21/20; A61F 2002/0068; B32B 2250/03; B32B 2250/20; B32B 2262/0253; B32B 2262/0276; B32B 2262/0284; B32B 2262/16; B32B 2307/718; B32B 2535/00; D10B 2509/00

(57) ABSTRACT

The present invention relates to an implantable device, in particular for parietal surgery, comprising a warp-knitted fabric, said knitted fabric comprising:
a first knitted layer comprising a monofilament yarn A knitted with tricot stitches,
a second knitted layer comprising a monofilament yarn B knitted with tricot stitches,
a third knitted layer with a monofilament yarn C knitted with tricot stitches, and
said warp-knitted fabric comprises a knitted bottom and comprises gripping knitted loops projecting from said knitted bottom and inclined towards said knitted bottom. The present invention also relates to a method for manufacturing such a device.

### CROSS REFERENCE TO RELATED APPLICATIONS

This application is the U.S. National Stage, under 35 U.S.C. § 371, of International Application No. PCT/EP2024/058149, filed Mar. 26, 2024, which claims the benefit of FR2302903, filed Mar. 27, 2023, the contents of each of which are hereby incorporated by reference herein.

### TECHNICAL FIELD

The present application relates to an implantable device, in particular for parietal surgery, in particular for the repair of hernias.

The present application also relates to a method for manufacturing such an implantable device.

### STATE OF THE ART

It is known to treat parietal insufficiencies (hernias and eventrations in particular) to use a textile prosthesis to mechanically reinforce the deficient abdominal or inguinal wall. In order to maintain the reinforcing textile prosthesis on the abdominal or inguinal wall, the surgeon uses sutures and/or staples. Before placing the sutures or staples, it is quite common for the surgeon to correctly reposition the prosthesis by moving it laterally so that it can cover the inguinal orifices. The purpose of the sutures is to maintain the prosthesis in its final implantation position selected by the surgeon, while it is colonized by native scar tissue which is the consequence of a fibroblastic reaction. However, these fixations cause pain and are uncomfortable for the patient.

To reduce the pain caused by sutures, it has been proposed to integrate bioadhesives (that is to say, those activated by the water contained in the tissues of the human body) into the prostheses. These bioadhesives do not allow satisfactory repositioning of the prosthesis when it is put in place using a TAPP (TransAbdominal PrePeritoneal) or TEP (Totally Extra-peritoneal) surgical technique. Either because they are too adherent and it is difficult or even impossible to consider lateral displacement (for example, this is the case with the Lifemesh™ prosthesis from the company Lifebond, which includes an adhesive that fixes immediately upon contact with the tissues), or because the adhesion is too weak and does not allow the position given by the surgeon to be maintained in the long term, even if lateral displacement of the prosthesis is easier. Control of sufficient adhesion to allow the prosthesis to be maintained in its implantation position in the long term, but not too excessive to allow lateral repositioning during its placement, cannot be achieved with a bioadhesive.

Thus, provision is made of an implantable device comprising a knitted fabric from which rectilinear spikes project, provided at their free ends with a fused head having a larger cross-section than the body of a spike. This type of device is marketed under the brand name ProGrip™ by the Medtronic company and its manufacturing technique is described in patent EP 1.274.892B1. This prosthesis comprises spikes which do not allow lateral repositioning since the spikes penetrate the muscle tissues when the prosthesis is put in place. When the prosthesis is repositioned, the spikes tear the muscle tissues and lose their adhesive capacity. This prosthesis also has a major disadvantage compared to the disadvantages previously mentioned. Indeed, for placement, the prosthesis is rolled up on itself to be passed through a trocar, but it has the particularity of gripping on itself.

This disadvantage forces the surgeon to force its unwinding for its insertion, which causes a degradation of the spikes. The operating time using a TAPP or TEP surgical technique is then significantly extended. A solution proposed to this problem was to cover the gripping spikes with a water-soluble film that dissolves almost immediately after implantation. This solution described in patent FR2949687B1 has the disadvantage that the method is more complex and lengthy since an additional step of adding a film is necessary. Once the water-soluble film is dissolved in the body, the spikes enter the abdominal fat and muscle tissue. The spikes that penetrate the muscle wall can come into contact with nerves and cause pain in the patient.

There is thus a need for an implantable device, particularly for parietal surgery, which allows immediate adhesion to the abdominal or inguinal wall, which is repositionable, does not migrate laterally after implantation, preserves patient comfort (particularly by avoiding penetration into the muscle of the abdominal wall in order to limit pain), and finally can be rolled and unrolled without risk of degradation of its mechanical properties.

Finally, there is a need for a manufacturing method for such an implantable device which is simple and reliable.

The present application relates to an implantable device that does not require fixation by means of a suture or synthetic glue, limits pain without penetrating muscle tissue, is easily unrolled after passing through a trocar, is easily repositioned laterally without damaging the tissues to allow its precise placement on the surgical site by a TAPP or TEP technique in order to cover the hernial orifices, and finally does not migrate laterally in the long term.

### DISCLOSURE

The present application relates, according to a first aspect, to an implantable device, in particular for parietal surgery, characteristically comprising a warp-knitted fabric, said knitted fabric comprising:
a first knitted layer comprising a monofilament yarn A knitted with tricot stitches,
a second knitted layer comprising a monofilament yarn B knitted with tricot stitches,
a third layer knitted with a monofilament yarn C knitted with tricot stitches, and
said warp-knitted fabric comprises a knitted bottom and comprises gripping knitted loops projecting from said knitted bottom and inclined towards said knitted bottom.

Advantageously, two knitted layers, in particular the first and second knitted layers, form at least in part the knitted bottom of the warp-knitted fabric, while the remaining knitted layer, in particular the third knitted layer, is regularly anchored in the knitted bottom and forms small knitted loops, closed, and projecting from said knitted bottom while being inclined relative to the latter.

Advantageously, the knitted bottom comprises, in particular is formed of, the first and second knitted layers, and optionally comprises, in particular is formed of, at least in part the third knitted layer.

Advantageously, the knitted loops comprise, in particular are formed, at least in part from the third knitted layer, and optionally from the first and/or second knitted layer(s).

Advantageously, at least one gripping and inclined knitted loop, in particular the gripping and inclined knitted loops, is (are each) a mesh loop, in particular formed by a needle.

Advantageously, at least one gripping and inclined knitted loop, in particular the gripping and inclined knitted loops, is/are not one or more knitted float(s).

Advantageously, the inclined and gripping knitted loops of the warp-knitted fabric according to the application offer numerous advantages. These gripping loops allow lateral repositioning of the implantable device because they penetrate the fatty tissue but without penetrating, or at least not significantly, the muscle tissue. This arrangement considerably improves patient comfort.

Advantageously, it has been observed that the gripping knitted loops penetrate fatty tissue but do not retain a large amount of fatty tissue when the loops are removed from the fatty tissue, allowing the implantable device to be repositionable.

Advantageously, the gripping loops are inclined, allowing immediate adhesion without penetrating the muscle tissue. The inclination of the gripping loops also prevents the implantable device from migrating laterally, that is to say from translating, along the abdominal wall for example.

The implantable device can also be rolled up onto itself for insertion into a trocar, then easily unrolled without the inclined gripping loops catching on the knitted structure and hindering unrolling. The implantable device can therefore be rolled up and unrolled without risk of damage to the warp-knitted fabric.

Finally, the inclined gripping loops are formed by knitting and therefore obtained inclined at the exit of the knitting machine. The gripping members are therefore not formed during an additional operation carried out on the warp-knitted fabric after knitting at the risk of degrading the mechanical resistance of the warp-knitted fabric. The gripping and inclined loops formed by knitting are therefore very reproducible from one loop to another: their rigidity, their dimensions, their number and their distribution are therefore related to the knitting method with monofilament yarns. This arrangement allows to obtain immediate adhesion, a capacity to be repositionable, and to be moved transversely without penetrating into the muscle tissues.

Preferably, the warp-knitted fabric comprises a first face and a second face that are substantially opposite.

Preferably, the knitted bottom extends between said first face and said second face of the warp-knitted fabric.

Preferably, the gripping and inclined knitted loops project from the first face of the knitted bottom of the warp-knitted fabric.

Preferably, the warp-knitted fabric does not comprise inclined gripping loops extending along its second face.

Preferably, the warp-knitted fabric comprises a first lateral edge and a second lateral edge, in particular substantially parallel to each other.

A first lateral edge, respectively a second lateral edge, may be straight or not, for example wavy or convex or concave.

Preferably, the warp-knitted fabric comprises an upper edge and a lower edge.

In particular, the first lateral edge, respectively the second lateral edge, extends between the upper edge and the lower edge.

The warp-knitted fabric can have any shape, for example rectangular or square.

Advantageously, since the warp-knitted fabric is a non-running knit, the surgeon can re-cut the warp-knitted fabric once removed from its sterilized packaging bag to adapt it to the parietal defect to be treated without the risk of the cut portion(s) fraying.

Advantageously, the warp-knitted fabric comprises a warp direction, and columns of stitches extending longitudinally in the warp direction.

Advantageously, the warp-knitted fabric comprises a weft direction, and rows of stitches extending longitudinally in the weft direction.

In particular, the weft direction is substantially perpendicular to the warp direction.

In this text, tricot stitches are understood to mean stitches, that are in particular closed or open, preferably closed, (that is to say formed by a given yarn) extending over at least two columns of stitches of the warp-knitted fabric or over at least two needles.

In this text, a knitted layer comprising a monofilament yarn is understood to mean that said layer is formed in part or entirely by said knitted monofilament yarn.

In this text, a knitted layer is understood to mean a knitted network formed by at least one yarn extending over the width and height of the warp-knitted fabric.

Advantageously, the monofilament yarn knitted into a layer is knitted in a repeat pattern that is repeated across the width of the warp-knitted fabric between the first and second lateral edges, and across the height of the warp-knitted fabric between the top and bottom edges.

In particular, a knitted layer is knitted with at least one yarn supported by a guide bar working on a knitting machine.

Advantageously, the warp-knitted fabric is therefore knitted with at least three different guide bars, in particular each guide bar has a movement (that is to say a stitch pattern) different from the movements (that is to say the stitch patterns) of the at least two other guide bars.

The warp-knitted fabric can be knitted on a warp or rachel knitting machine.

In particular, the warp-knitted fabric is knitted with at least three different guide bars each supporting at least one monofilament yarn.

Advantageously, the warp-knitted fabric comprises, in particular is formed of, three monofilament yarns A, B and C each knitted according to different repeat patterns. In particular, a repeat pattern corresponds to the movement of a guide bar on the needles of the knitting machine.

### Monofilament Yarn(s) A and/or B and/or C

The monofilament yarn(s) is/are in one or more biocompatible materials, that is to say suitable for implantation in a living organism, human or animal organism, for a period of a few hours to several months or years.

The monofilament yarn(s) A and/or B and/or C may be made of one or more materials selected from:
a list I comprising non-(bio)resorbable materials, preferably the non-(bio)resorbable materials comprise: non-(bio)resorbable polyesters, such as polyethylene terephthalate (PET), and polybutylene terephthalate (PBT); polyamides; polyetheretherketon (PEEK); polyolefins, such as polypropylene and polyethylene; and a mixture thereof; more preferably polyolefins, and non-(bio)resorbable polyesters;
a list II comprising bioresorbable materials, preferably the bioresorbable materials comprise: polylactic acid (PLA, PDLA, PLLA), polyglycolic acid (PGA), oxidized cellulose, polycaprolactone (PCL), polydioxanone (PDO), polytrimethylenecarbonate (TMC), polyvinyl alcohol (PVA), polyhydroxyalkanoate (PHA), Poly4-Hydroxybutyrate (P4HB), P4HB (poly4-Hydroxybutyrate) and P3HB (poly3-Hydroxybutyrate) block copolymers, lactic acid or glycolic acid and caprolactone copolymers, trimethylenecarbonate and lactic acid copolymers, in particular PLA, PLLA, PDLA; copolymers of the latter, or a mixture thereof, more preferably polylactic acid (PLA, PLLA, PDLA), polyglycolic acid (PGA), and triethylene carbonate and lactic acid (PLA, PLLA, PDLA) copolymers, or a mixture thereof.

Preferably, yarn A and/or yarn B is/are in one or more materials selected from list I.

Preferably, the yarn C is in one or more materials selected from list II.

Preferably, at least a portion of the inclined gripping loops, more preferably the inclined gripping loops, is/are bioresorbable. When the inclined and gripping loops are bioresorbable, they are formed from one or more bioresorbable material(s).

Advantageously, the inclined gripping loops ensure their immediate and repositionable adhesion function for a time allowing the scar fibrosis to take over for the definitive attachment of the implantable device to the abdominal wall to be treated. The gripping loops are therefore resorbed over time and disappear. Only the first and second knitted layers remain as permanent mechanical reinforcement.

The monofilament yarn(s) A and/or B and/or C each preferably has a diameter comprised between 50 μm and 300 μm, more preferably comprised between 80 μm and 200 μm, more preferably comprised between 80 μm and 150 μm.

The monofilament yarn(s) A and/or B each has/have a diameter comprised between 50 μm and 200 μm, for example of the order of 100+/−10 μm.

In particular, the monofilament yarn C has a diameter comprised between 100 μm and 300 μm, for example of the order of 150+/−10 μm. It is understood in this text that a parameter is comprised between x and y, that the values x and y are comprised in the interval from x to y, and therefore that said parameter can be equal to x or y.

The monofilament yarn(s) A and/or B and/or C may be one or more multifilament yarn(s), in particular in one or more materials selected from list I and/or II.

The multifilament yarn(s) preferably has/have a linear mass comprised between 50 dtex and 400 dtex, more preferably comprised between 60 dtex and 300 dtex, preferably comprised between 60 dtex and 220 dtex.

In particular, the monofilament yarn(s) A and/or B each has/have a linear density comprised between 60 dtex and 300 dtex, for example of the order of 70+/−10 dtex.

In particular, the monofilament yarn C has a linear density comprised between 60 dtex and 300 dtex, for example of the order of 220+/−15 dtex.

In one embodiment, the warp-knitted fabric consists essentially of the first, second, and third knitted layers.

It is understood that the warp-knitted fabric is essentially made up of the first, second, and third knitted layers that said knitted layers represent at least 80% by mass, preferably at least 90% by mass, more preferably at least 95% by mass, preferably approximately 100% by mass, of the total mass of the warp-knitted fabric.

For example, when the three knitted layers represent at least 80% by mass of the warp-knitted fabric, said warp-knitted fabric may comprise one or more other yarn(s) but not forming a knitted layer, that is to say repeated over the width and height of the knit.

In this text, a gripping loop is understood to mean a loop configured to grip the abdominal/inguinal wall to be treated, particularly fatty tissue.

In this text, an inclined loop is understood to mean that a loop does not extend substantially vertically from the knitted bottom, but is inclined towards said knitted bottom, and is in particular oriented in a given direction, for example in a direction F1 or F2 or F3 or F4, more particularly corresponding to the direction of placement of the implantable device in the caudal direction of the patient being treated.

Advantageously, the inclination of the loops allows to avoid migration of the implantable device in the caudal direction when the patient is in a standing position, that is to say in the direction in which the inclined loops are oriented.

Preferably, the warp-knitted fabric comprises a direction F1 and a direction F2 opposite, and substantially parallel to the warp direction or to the direction of the columns of stitches of the warp-knitted fabric. More preferably, the direction F1 extends from the lower edge of the warp-knitted fabric to the upper edge of the warp-knitted fabric, and the direction F2 extends from the upper edge of the warp-knitted fabric to the lower edge of the warp-knitted fabric.

Preferably, the warp-knitted fabric comprises a direction F3 and a direction F4 opposite, and substantially parallel to the weft direction or to the direction of the rows of stitches.

More preferably, the direction F3 extends from the second lateral edge of the warp-knitted fabric to the first lateral edge of the warp-knitted fabric, and the direction F4 extends from the first lateral edge of the warp-knitted fabric to the second lateral edge of the warp-knitted fabric.

In one embodiment, a part (in particular between one-third and one-half of the total gripping loops) of the gripping and inclined loops of the warp-knitted fabric are oriented in direction F4, and/or a part (in particular between one-third and one-half of the total gripping loops) of the gripping and inclined loops of the warp-knitted fabric are oriented in the direction F3.

Preferably, a gripping inclined loop, in particular each of the gripping inclined loops, comprises a distal end and a loop foot, more preferably comprises an axis of symmetry S passing through said distal end and separating the loop into first and second lateral portions symmetrical to each other with respect to said axis S.

Preferably, the gripping knitted loops are knitted loops, that is to say meshed loops (that is to say each formed by knitting a needle).

Preferably, the gripping knitted loops are each formed from a closed knitted stitch.

It is understood by a gripping inclined loop oriented in the direction F4, respectively F3, that the distal end of said loop is oriented opposite the second, respectively the first, lateral edge.

The warp-knitted fabric may comprise gripping inclined loops all oriented in the same direction (F3 or F4 for example); or gripping inclined loops oriented in different directions; or first gripping inclined loops oriented in a given direction F (F3 or F4 for example) and second gripping inclined loops oriented in a given direction F′ opposite to the direction F (F3 or F4 for example), said first and second gripping loops are repeated between the first and second lateral edges of the knitted fabric and between the upper and lower edges of the knitted fabric in an alternating manner.

The warp-knitted fabric may be coated on its second face, in particular its second face not comprising inclined gripping loops, with a bioresorbable or non-bioresorbable coating.

In an alternative embodiment, the warp-knitted fabric comprises a first lateral edge and a second lateral edge, said first and second lateral edges extend in the warp direction of the warp-knitted fabric, and said warp-knitted fabric comprises at least one set comprising first gripping loops oriented towards the first lateral edge and second gripping loops oriented towards the second lateral edge, preferably said first loops and second loops of said set are arranged, in particular alternately, in the warp direction.

In this text, a loop oriented towards the first, respectively second, lateral edge is understood to mean that the distal end of said loop is oriented opposite the first, respectively second, lateral edge of the warp-knitted fabric.

Advantageously, the orientation of the inclined loops is alternated towards the first lateral edge then towards the second lateral edge of the warp-knitted fabric according to a determined sequence extending in the warp direction of the warp-knitted fabric.

Advantageously, the first and second lateral edges of the warp-knitted fabric are positioned in the cranial/caudal direction of the patient being treated so that the gripping loops are oriented alternately towards a first lateral side then towards a second lateral side of the patient. This alternating orientation of the loops allows to avoid migration in a horizontal direction when the patient is in a standing position.

Advantageously, the alternating arrangement in the warp direction of the first and second gripping inclined loops allows to avoid migration of the implantable device after implantation in a horizontal direction of the patient, corresponding to the weft direction of the warp-knitted fabric (in particular according to either one of the directions F3 and F4).

The first loops may be alternated with the second loops in a determined sequence, for example by alternating in the warp direction, a first loop with a second loop, or by alternating a first loop with two or more second loops or conversely by alternating a second loop with two first loops. Preferably, the first and second loops are alternated so that a first loop is alternated with a second loop and so on. The regular alternation of the first and second loops allows to improve the stabilization of the warp-knitted fabric at the implantation site.

Advantageously, the angle formed between the axis of symmetry S of a gripping inclined loop and the direction of the first, respectively second, lateral edge is comprised between 20° and 80°, preferably is comprised between 30° and 60°, preferentially is comprised between 35° and 55°, for example is of the order of 45°+/−5°.

Preferably, said at least one set of (first and second) inclined and alternately oriented loops extends between the upper and lower edges of the warp-knitted fabric, in particular over the entire height of the warp-knitted fabric.

Said first and second lateral edges may be parallel to each other and/or parallel to the warp direction of the warp-knitted fabric.

Advantageously, since the loops are already inclined, the surgeon can accentuate the inclination of the loops at the surgical site during implantation. This arrangement improves the blocking of caudal vertical migration.

In an alternative embodiment, the warp-knitted fabric comprises a plurality of sets of loops, inclined and oriented, disposed between the first and second lateral edges.

Advantageously, the warp-knitted fabric comprises several sets of inclined and oriented loops, each set comprising first loops inclined and oriented towards the first lateral edge and second loops inclined and oriented towards the second lateral edge, preferably said first loops and second loops of said set are arranged, in particular alternately, in the warp direction.

Advantageously, the sets of inclined and oriented loops are arranged side by side between the first and second lateral edges.

In an alternative embodiment, the first knitted layer comprises first portions of tricot stitches, in particular each knitted according to a knitted pattern M1, each extending over at least three columns of stitches, and second portions of tricot stitches, in particular each knitted according to a knitted pattern M2, each extending over at least three columns of stitches, the first portions of tricot stitches are alternated with the second portions of tricot stitches, and the second portions of tricot stitches are knitted in an inverted manner with respect to the first portions of tricot stitches or the second knitted pattern M2 is inverted with respect to the first knitted pattern M1.

Preferably, the first, respectively second, portions of tricot stitches each extend over three rows of stitches.

In an alternative embodiment, the first knitted layer comprises first portions of tricot stitches, in particular each knitted according to a knitted pattern M1, each extending over at least three columns of stitches, and second portions of tricot stitches, in particular each knitted according to a knitted pattern M2, each extending over at least three columns of stitches, the first portions are alternated with the second portions. The first portions of tricot stitches are each in the shape of a lying V open towards the second lateral edge (“<”), in particular corresponding to the first knitted pattern M1, while the second portions of tricot stitches are each in the shape of a lying V open towards the first lateral edge (“>”), in particular corresponding to the second knitted pattern M2.

In a variant, the first portions of tricot stitches are alternated with the second portions of tricot stitches in the warp direction of the warp-knitted fabric, in particular the first yarn forms first portions of tricot stitches and second portions of tricot stitches which are alternated in the warp direction.

In an alternative embodiment, the second knitted layer comprises third portions of tricot stitches, in particular each knitted according to a knitted pattern M3, each extending over at least three columns of stitches, and fourth portions of tricot stitches, in particular each knitted according to a knitted pattern M4, each extending over at least three columns of stitches, the third portions of tricot stitches are alternated with the fourth portions of tricot stitches, and the fourth portions of tricot stitches are knitted in an inverted manner relative to the third portions of tricot stitches, or the third knitted pattern M3 is inverted relative to the fourth knitted pattern M4.

In a variant, the third portions of tricot stitches are alternated with the fourth portions of tricot stitches in the warp direction of the warp-knitted fabric, in particular the second yarn forms third portions of tricot stitches and fourth portions of tricot stitches which are alternated in the warp direction.

In a variant, the third, respectively fourth, portions of tricot stitches each extend over three rows of stitches.

In an alternative embodiment, the second knitted layer comprises third portions of tricot stitches, in particular each knitted according to a knitted pattern M3, each extending over at least three columns of stitches, and fourth portions of tricot stitches, in particular each knitted according to a knitted pattern M4, each extending over at least three columns of stitches, the third portions of tricot stitches are alternated with the fourth portions of tricot stitches, and the third portions of tricot stitches are each in the shape of a lying V open towards the first lateral edge (“>”), in particular corresponding to the third knitted pattern M3, while the fourth portions of tricot stitches are each in the shape of a lying V open towards the second lateral edge (“<”), in particular corresponding to the fourth knitted pattern M4.

In an alternative embodiment, the third knitted layer comprises fifth portions of tricot stitches, in particular each knitted according to a knitted pattern M5, each extending over at least two columns of stitches, and sixth portions of tricot stitches, in particular each knitted according to a knitted pattern M6, each extending over at least two columns of stitches, the fifth portions of tricot stitches and the sixth portions of tricot stitches are alternated, and the fifth portions of tricot stitches are each in the shape of a lying V open towards the first lateral edge (“>”), in particular corresponding to the third knitted pattern M5, while the sixth portions of tricot stitches are each in the shape of a lying V open towards the second lateral edge (“<”), in particular corresponding to the sixth knitted pattern M6.

Alternatively, the fifth portions of tricot stitches are offset by at least one column of stitches from the sixth portions of tricot stitches.

In a variant, the third knitted layer comprises fifth portions of tricot stitches, in particular each knitted according to a knitted pattern M5, each extending over at least two columns of stitches, and sixth portions of tricot stitches, in particular each knitted according to a knitted pattern M6, each extending over at least two columns of stitches, the fifth portions of tricot stitches and the sixth portions of tricot stitches are alternated, and the fifth portions of tricot stitches are each in the shape of a lying V open towards the first lateral edge (“>”), in particular corresponding to the knitted pattern M5, while the sixth portions of tricot stitches are each in the shape of a lying V open towards the second lateral edge (“<”), in particular corresponding to the knitted pattern M6.

Preferably, the fifth portions of tricot stitches are offset from the sixth portions of tricot stitches by at least one column of stitches.

Preferably, the fifth and sixth knitted sections are arranged one above the other between the upper and lower edges of the warp-knitted fabric.

Preferably, the warp-knitted fabric comprises several fifth and sixth sections knitted alternately in the warp direction, in particular between the upper and lower edges of the warp-knitted fabric.

Preferably, the warp-knitted fabric comprises a plurality of fifth and sixth sections knitted alternately in the warp direction, in particular the alternation repeating between the upper and lower edges of the warp-knitted fabric.

In an alternative embodiment, a first portion of tricot stitches, and a third portion of tricot stitches, are knitted on three columns of common stitches, and in particular on three rows of common stitches.

In an alternative embodiment, a second portion of tricot stitches and a fourth portion of tricot stitches are spaced at least one column of stitches apart.

In an alternative embodiment, the warp-knitted fabric comprises:
a first knitted section comprising tricot stitches, said first knitted section extending over at least three columns of stitches, in particular and extending over at least three rows of stitches, and is knitted with at least yarns A and B, and optionally yarn C, and
a second knitted section comprising tricot stitches, said second knitted section extending over at least three columns of stitches, in particular and extending over at least three rows of stitches, and is knitted with at least yarns A and B, and optionally yarn C, and
the second knitted section is spaced from the first knitted section by a through orifice, in particular having at least one dimension greater than or equal to 3 mm.

The first section is formed by a portion of the first knitted layer, a portion of the second knitted layer, and finally a portion of the third knitted layer.

The first knitted section extends between the first and second faces of the warp-knitted fabric.

Preferably, the first knitted section and the second knitted section are arranged side by side in the weft direction of the warp-knitted fabric.

Preferably, the first knitted section and the second knitted section are knitted over at least three rows of common stitches.

Said at least one dimension of an orifice is preferably measured on the warp-knitted fabric at rest, that is to say without exerting any tension on the latter, using a microscope or a graduated ruler.

Said at least one dimension is preferably any dimension of an orifice, and in particular may be the width, the length, the diameter, or else the distance between the two most distant points taken on the periphery of the orifice.

Preferably, the first knitted section and the second knitted section are spaced at least one column of stitches apart.

Preferably, the warp-knitted fabric comprises a plurality of first knitted sections and a plurality of second knitted sections, said first and second knitted sections being alternated in the weft direction of the warp-knitted fabric, in particular are repeatedly arranged between the first and second lateral edges.

In an alternative embodiment, the first knitted section is in knitted connection with the second knitted section, preferably via a third knitted section, said third knitted section comprising tricot stitches, said third knitted section extends over at least three columns of stitches, in particular and extending over at least three rows of stitches, and is knitted with at least yarns A and B, and optionally yarn C.

Preferably, the first knitted section is in knitted connection with the second knitted section also via a fourth knitted section, said fourth knitted section comprising tricot stitches extending over at least three columns of stitches, in particular and extending over at least three rows of stitches, and knitted with at least yarns A and B, and optionally yarn C.

Preferably, the warp-knitted fabric comprises several through openings each having at least one dimension greater than or equal to 3 mm.

Preferably, a through opening is delimited, at least in part, by the first, second, third and fourth knitted sections.

Preferably, the third and fourth knitted sections are arranged one above the other between the upper and lower edges of the warp-knitted fabric.

Preferably, the warp-knitted fabric comprises a plurality of third and fourth sections knitted alternately in the warp direction, particularly between the upper and lower edges of the warp-knitted fabric.

Preferably, the warp-knitted fabric comprises several through openings each having at least one dimension greater than or equal to 3 mm, each of the through openings being delimited at least in part by first, second, third and fourth knitted sections.

In an alternative embodiment, the warp-knitted fabric comprises a gripping inclined loop formed at least in part by a portion of a monofilament yarn selected from yarns A, B or C, said portion of monofilament yarn is knitted in at least one of the first, second, third and fourth knitted sections.

Advantageously, said at least one gripping and inclined loop is formed by a portion of the monofilament yarn C.

In an alternative embodiment, at least two, preferably at least three, more preferably each, of the first, second, third and fourth knitted sections comprises/comprise a gripping and inclined loop formed by a portion of a monofilament yarn selected from yarns A, B or C, in particular yarn C.

In a variant, the warp-knitted fabric comprises several through openings, at least one of which through openings, in particular each of said through openings, has a dimension greater than or equal to 3 mm, said through opening is surrounded at least in part by at least two, preferably at least four, gripping and inclined loops, each gripping and inclined loop being formed by a portion of monofilament yarn, in particular a monofilament yarn C, more particularly said through opening is surrounded by:
a gripping and inclined loop O1 arranged in the first knitted section, and in particular oriented towards the second lateral edge, more particularly in the direction F4; and/or
a gripping and inclined loop O2 arranged in the second knitted section, and in particular oriented towards the second lateral edge, more particularly in the direction F4; and/or
a gripping and inclined loop O3 arranged in the third knitted section, and in particular oriented towards the first lateral edge, more particularly in the direction F3; and/or
a gripping and inclined loop O4 arranged in the fourth knitted section, and in particular oriented towards the first lateral edge, more particularly in the direction F3.

In an alternative embodiment, at least one loop selected from said gripping inclined loops, in particular several or each of the gripping and inclined loops, is/are knitted with the monofilament yarn C, and said, or each of said, gripping inclined loop(s) is arranged between a first base stitch and a second base stitch knitted with said monofilament yarn C, and the warp-knitted fabric comprises a first blocking stitch knitted with yarn A or B, preferably yarn B, and superimposed with the first base stitch, and optionally a second blocking stitch knitted with yarn A or B, preferably yarn B, and superimposed with the second base stitch.

Advantageously, the first base stitch and the first blocking stitch, possibly the second base stitch and the second blocking stitch, are in the knitted section (in particular first or second or third or fourth) comprising the gripping inclined knitted loop.

Advantageously, when the warp-knitted fabric is relaxed as it leaves the knitting machine, the portions of yarn forming the inclined, oriented, gripping loops retract and each form a loop.

Advantageously, at the outlet of the knitting machine, for a given gripping inclined loop, a stitch is formed between the first and second base stitches blocked by the first and second blocking stitches, said stitch unravels (that is to say it is released from the needle on which said stitch was knitted) then forms a portion of looped monofilament yarn extending between the first and second base stitches, said portion forming the gripping and inclined loop.

Advantageously, the blocking stitches allow the gripping and inclined knitted loops to maintain their loop shape when leaving the knitting machine and prevent them from forming floats when untwisting.

Preferably, the first, or second, blocking stitch and the first, or second, base stitch are knitted on the same needle and/or interlaced.

Preferably, a gripping inclined loop, or each of said gripping inclined loop(s), is arranged between a first base stitch and a second base stitch knitted with said monofilament yarn C, and the warp-knitted fabric comprises a first blocking stitch knitted with yarn A or B, preferably yarn B, and superimposed with the first base stitch, and optionally a second blocking stitch knitted with yarn A or B, preferably yarn B, and superimposed with the second base stitch, and the first base stitch and/or the first blocking stitch, and/or the second base stitch and/or the second blocking stitch, is/are arranged on a column of stitches adjacent to the column of stitches comprising said gripping knitted loop. This arrangement helps to prevent the gripping knitted loop from untwisting and forming a float.

In an alternative embodiment, the knitting diagram of the first layer is: 5-4/5-6/5-4/8-9/8-7/8-9//.

This knitting diagram corresponds advantageously to the movement of the guide bar supporting at least yarn A, and knitting the first knitted layer.

In this text, all knitting diagrams are written in accordance with standard ISO 11676:2014 entitled “Textile machinery—Pattern discs and pattern chains for warp knitting machines—Terms and symbols”.

In an alternative embodiment, the knitting diagram of the second layer is: 1-0/2-3/1-0/4-5/3-2/4-5//.

This knitting diagram corresponds advantageously to the movement of the guide bar supporting at least yarn B, and knitting the second knitted layer.

In an alternative embodiment, the knitting diagram of the third layer is: 4-5/3-2/4-5/1-0/2-3/1-0//.

This knitting diagram corresponds advantageously to the movement of the guide bar supporting at least yarn C, and knitting the third knitted layer.

In an alternative embodiment, at least one loop selected from said gripping inclined loops, in particular several or each of the gripping inclined loops, is/are knitted with the monofilament yarn C, and said, or each of said gripping inclined loop(s) is knitted on a row of stitches n and on a column of stitches p, and the knitted yarns A and B are knitted so as to cross on either side of the gripping knitted loop: on said column of stitches p and on a row of stitches n+1 adjacent to the row of stitches n, and on said column of stitches p and on a row of stitches n−1 adjacent to the row of stitches n, p and n being whole numbers greater than or equal to 1.

Advantageously, the crossings of the knitted yarns A and B on either side of the gripping knitted loops promote their blocking and prevent the gripping knitted loops from untwisting and forming floats without gripping function.

In an alternative embodiment, the warp-knitted fabric comprises at least 40% by mass of one or more bioresorbable (co)polymer(s), in particular said bioresorbable (co)polymer(s) is/are selected from: polylactic acid (PLA), polyglycolic acid (PGA), oxidized cellulose, polycaprolactone (PCL), polydioxanone (PDO), polytrimethylenecarbonate (TMC), a copolymer of trimethylene carbonate (PTMC) and L-lactic acid, polyvinyl alcohol (PVA), polyhydroxyalkanoate (PHA), Poly4-Hydroxybutyrate (P4HB), P4HB and P3HB block copolymers, a mixture thereof, in particular selected from list II defined in the present text.

In one embodiment, the warp-knitted fabric comprises between 40% and 75%, in particular between 55% and 65%, of bioresorbable (co)polymer(s), in particular selected from list II defined in the present text.

In particular, the warp-knitted fabric comprises one or more monofilament yarn(s), in particular selected from yarns A, B and C, comprising one or more bioresorbable (co)polymer(s), in particular selected from list II mentioned above, said bioresorbable (co)polymer(s) representing at least 40% by mass of the total mass of the warp-knitted fabric.

Alternatively, the warp-knitted fabric comprises at least one yarn, in particular yarn C, comprising a copolymer of polytrimethylene carbonate (PTMC) and lactic acid, in particular L-lactic acid (PLLA).

Preferably, the mass fraction of lactic acid, in particular L-lactic acid, in said copolymer is greater than or equal to 60%, more preferably greater than or equal to 70%, in particular approximately 80% to within +/−5%.

Preferably, the mass fraction of trimethylene carbonate in said copolymer is less than or equal to 40%, more preferably less than or equal to 30%, in particular approximately 20% to within +/−5%.

Preferably, the copolymer of polytrimethylene carbonate (PTMC) and lactic acid is a block copolymer, in particular a triblock copolymer whose repeating unit comprises a central block and two side blocks arranged on either side of the central block, the central block comprises a trimethylene carbonate unit, and each side block comprises a lactic acid unit, in particular L-lactic acid.

In an alternative embodiment, the warp-knitted fabric comprises less than 60% by mass of one or more non-bioresorbable (co)polymer(s), in particular said non-bioresorbable (co)polymer(s) is/are selected from: (co)polymers of at least one olefin (for example polypropylene, polyethylene, a copolymer of propylene and ethylene), polyesters such as polyethylene terephthalate (PET) and polybutylene terephthalate (PBT), more particularly selected from list I defined in this text.

In particular, the warp-knitted fabric comprises one or more monofilament yarn(s), in particular selected from yarns A, B and C, comprising one or more non-(bio)resorbable (co)polymer(s), in particular selected from list I mentioned above, said non-resorbable (co)polymer(s) represent(s) less than 45% by mass of the total mass of the warp-knitted fabric, in particular said non-resorbable (co)polymer(s) is/are selected from: (co)polymers of at least one olefin (for example polypropylene, polyethylene, a copolymer of propylene and ethylene), polyesters such as polyethylene terephthalate (PET) and polybutylene terephthalate (PBT).

In one embodiment, the warp-knitted fabric comprises between 20% and 40%, in particular between 30% and 40%, of non-(bio)resorbable (co)polymer(s).

In an alternative embodiment, the warp-knitted fabric has a surface mass greater than or equal to 50 g/m² and less than or equal to 150 g/m².

In a preferred embodiment, the warp-knitted fabric has a surface mass comprised between 80 g/m² and 120 g/m², in particular of the order of 100 g/m² to +/−5 g/m².

In an alternative embodiment, at least one of the yarns selected among the yarns A, B and C has a diameter greater than or equal to 0.02 mm and less than or equal to 0.30 mm.

Preferably, the warp-knitted fabric comprises at least one monofilament yarn A having a diameter comprised between 0.08 mm and 0.30 mm, at least one monofilament yarn B having a diameter comprised between 0.08 mm and 0.30 mm, at least one monofilament yarn C having a diameter comprised between 0.08 mm and 0.30 mm.

In an alternative embodiment, the warp-knitted fabric comprises a number of stitches in the warp direction less than or equal to 6 per cm, and a number of stitches in the weft direction less than or equal to 6 per cm.

In a preferred embodiment, the warp-knitted fabric comprises a number of stitches in the warp direction comprised between 1 and 6 per cm, preferably between 2 and 5 per cm.

In a preferred embodiment, the warp-knitted fabric comprises a number of stitches in the weft direction comprised between 1 and 6 per cm, preferably between 2 and 5 per cm.

The present invention application relates, according to a second aspect, to a method for manufacturing an implantable device according to any one of the alternative embodiments with reference to the first aspect, comprising a step of knitting a warp-knitted fabric on a knitting machine comprising:
knitting a first knitted layer with a first guide bar supporting at least one monofilament yarn A and forming tricot stitches,
knitting a second knitted layer with a second guide bar supporting at least one monofilament yarn B and forming tricot stitches,
knitting a third knitted layer with a third guide bar supporting at least one monofilament yarn C and forming tricot stitches, and
the movement of said first, second, and third guide bars is determined so as to obtain a warp-knitted fabric comprising a knitted bottom and comprising gripping knitted loops projecting from said knitted bottom and inclined towards said knitted bottom.

The manufacturing method advantageously allows to obtain a warp-knitted fabric with inclined and gripping loops obtained by knitting, and therefore arranged spatially and in number in a manner determined by the stitch pattern and reproducible.

Advantageously, the warp-knitted fabric comprises gripping loops without the need for an additional step after knitting to melt portions of yarn and form gripping picots.

Preferably, the knitting machine is a warp or Rachel machine.

Preferably, the first and second guide bars are each yarned one full and three empty.

Preferably, the third guide bar is yarned two empty, one full, and one empty.

The schematic representation . . . I means for example three empty and one full. A dot (.) represents an empty yarn, and (I) represents a full yarn.

In a variant, at least one loop, selected from said gripping inclined loops, is knitted with the monofilament yarn C, and said loop is arranged between a first base stitch and a second base stitch, and the warp-knitted fabric comprises a first blocking stitch knitted with yarn A or B and superimposed with the first base stitch, and optionally a second blocking stitch knitted with yarn A or B and superimposed with the second base stitch.

In an alternative embodiment, the movement of the first guide bar, in particular the front bar, is: 5-4/5-6/5-4/8-9/8-7/8-9//, in particular with repetitive yarning of three empty guide bars followed by one full guide bar (schematic representation: . . . I).

In an alternative embodiment, the movement of the second guide bar, in particular of the intermediate bar, is: 1-0/2-3/1-0/4-5/3-2/4-5//in particular with repetitive yarning of three empty guide bars followed by one full guide bar (schematic representation: . . . I).

In an alternative embodiment, the movement of the third guide bar, in particular the rear bar, is: 4-5/3-2/4-5/1-0/2-3/1-0//in particular with repetitive yarning of two empty guide bars followed by one full guide bar and followed by one empty guide bar (schematic representation: (. . . I.).

The variant embodiments described with reference to the first aspect of the application may be combined with the variant embodiments with reference to the second aspect of the application, independently of one another.

### DESCRIPTION OF THE DRAWINGS

The application will be better understood upon reading the following description of an exemplary embodiment of the application given without limitation, with reference to the appended drawings, in which:

FIG. 1 schematically shows, seen from the front, a first example of an implantable device according to the application,

FIG. 2 schematically shows, seen from the side, the first example of an implantable Figure shown in FIG. 1;

FIG. 3 schematically shows a top view of the warp-knitted fabric with a gripping inclined loop oriented towards the first lateral edge and a gripping inclined loop oriented towards the second lateral edge;

FIG. 4 shows the knitting diagram of the warp-knitted fabric of the first example of implantable device according to FIGS. 1 to 3.

### DESCRIPTION OF THE EMBODIMENTS

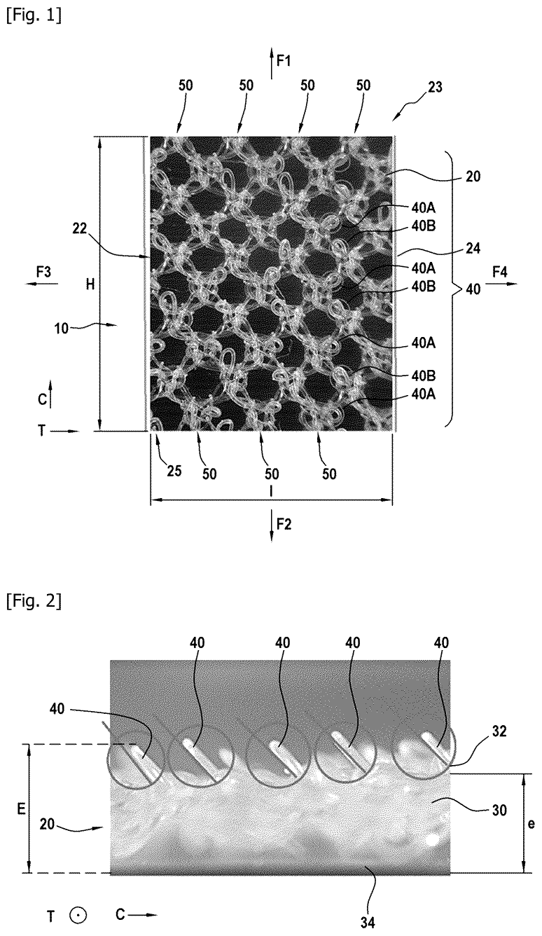

The implantable device 10 shown in FIGS. 1 and 2 comprises a warp-knitted fabric 20, said warp-knitted fabric 20 comprising a knitted bottom 30 having substantially opposite first and second faces 32, 34, and comprises gripping knitted loops 40 projecting from the first face 32 of the knitted bottom 30 and inclined towards said knitted bottom 30. The warp-knitted fabric 20 comprises a first lateral edge 22 and a second lateral edge 24, said first and second lateral edges 22, 24 extending in the warp direction C of the warp-knitted fabric. The warp-knitted fabric 20 comprises several sets 50 of inclined gripping loops 40, in particular with an alternating orientation in the warp direction C towards the first lateral edge 22 and towards the second lateral edge 24. Advantageously, each set 50 comprises first inclined gripping loops 40A which are oriented towards the first lateral edge 22, and second inclined gripping loops 40B which are oriented towards the second lateral edge 24. Advantageously, the first loops 40A and the second loops 40B of each set 50 extend so as to have an alternating orientation in the warp direction C of the knitted fabric 20.

The warp-knitted fabric 20 thus comprises several sets 50 of inclined loops 40, and in particular oriented in a determined manner, arranged between the first and second lateral edges 22, 24.

Preferably, the first and second loops 40A, 40B have an alternating orientation such that a first loop 40A has an orientation which is alternated with a second loop 40B and so on in the warp direction C. The alternation of the orientations of the first and second loops 40A, 40B could be different, for example a first loop 40A is oriented towards the first lateral edge 22 while two or more second loops 40B are oriented towards the second lateral edge 24, or two or more first loops 40A are oriented towards the first lateral edge 22 for a second loop 40B which is oriented towards the second lateral edge 24. The advantage is to obtain a regular alternation of the orientations of the first and second loops allowing to stabilize the lateral movements of the warp-knitted fabric 20 placed in contact with the adipose tissues.

Preferably, the sets 50 of inclined loops 40, and in particular oriented, extend between the upper and lower edges 23, 25 of the warp-knitted fabric 20, in particular over the entire height H of the warp-knitted fabric 20, and over the entire width I of the knitted fabric 20.

Preferably, the first and second lateral edges 22, 24 are parallel to each other and parallel to the warp direction C of the warp-knitted fabric 20.

FIG. 2 shows the inclination towards the knitted bottom 30 of the gripping loops 40.

The knitted bottom 30 has a thickness e less than the total thickness E. The thickness e of the knitted bottom 30 corresponds to the distance separating the first and second faces 34 and 32. The thickness E corresponds to the distance separating the first face 34 from the distal ends of the gripping loops 40.

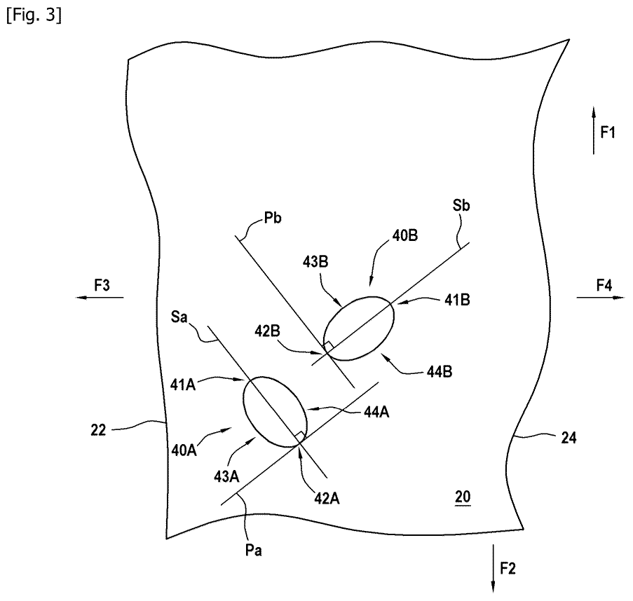

FIG. 3 schematically shows the warp-knitted fabric 20 seen from the front and slightly in perspective. Only a first gripping loop 40A and a second gripping loop 40B are shown for reasons of simplification.

In particular, each gripping inclined loop (40A, 40B) comprises a distal end (41A, 41B) and a loop foot (42A, 42B). The distal end 41A is oriented towards the first edge 22, and therefore in the direction F3, while the distal end 41B is oriented towards the second edge 24, and therefore in the direction F4.

The gripping inclined loop 40A comprises an axis of symmetry Sa passing through said distal end 41A and substantially perpendicular to the axis Pa tangent to the loop foot 42A.

The axis of symmetry Sa separates the loop 40A into first and second symmetrical lateral parts (43A, 44A).

The gripping inclined loop 40B comprises an axis of symmetry Sb passing through said distal end 41B and substantially perpendicular to the axis Pb tangent to the loop foot 42A. The axis of symmetry Sb separates the loop 40B into first and second symmetrical lateral parts (43B, 44B).

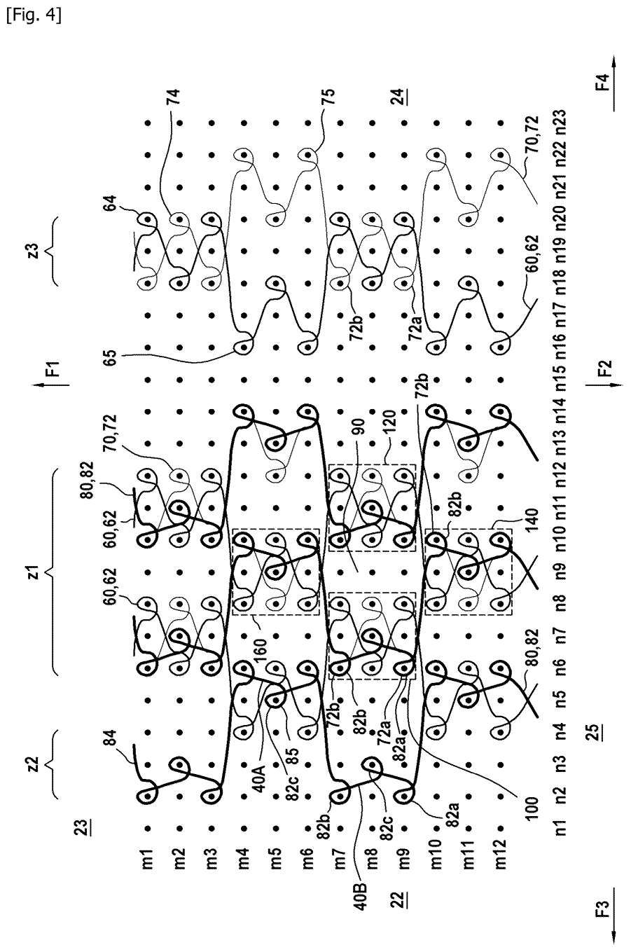

FIG. 4 shows the stitch patterns of the different yarns knitted in the warp-knitted fabric 20 using a stitch grid (each row of stitches representing the position of the knitting needles when making the corresponding row of stitches) comprising from m1 to m12 rows of stitches and from n1 to n23 columns of stitches.

Zone 1 of FIG. 3 represents the preferred stitch patterns of the three types of yarns A, B and C implemented in the warp-knitted fabric, with two repeats per yarn to understand the knitted arrangement of the warp-knitted fabric 20. Zone 3 represents in particular the stitch patterns of yarns A and B. Zone 2 represents in particular the stitch pattern of yarn C.

Advantageously, zone 1 comprises, represented twice, and knitted repeatedly over the entire width I of the knitted fabric 20 between its first and second lateral edges 22, 24 and over the entire height H of the knitted fabric 20 between its upper 23 and lower 25 edges:
a monofilament yarn A 62 so as to form a first knitted layer 60 comprising tricot stitches;
a monofilament yarn B 72 so as to form a second knitted layer 70 comprising tricot stitches; and
a monofilament yarn C 82 so as to form a third knitted layer 80 comprising tricot stitches.

Zones 2 and 3 are each shown with fewer yarns to better understand the stitch patterns for each yarn type. Zone 2 is thus focused on the knitted yarn C 82, and zone 3 is focused on the knitted yarns A and B (62,72) without the yarn C 82.

Advantageously, the first knitted layer 60 comprises:
first portions 64 of tricot stitches each extending over three columns of stitches and over three rows of stitches, each in the shape of a lying V (“<”) open towards the second lateral edge 24, and
second portions 65 of tricot stitches each extending over three columns of stitches and over three rows of stitches, each in the shape of a lying V (“>”) open towards the first lateral edge 22,
the second portions of tricot stitches 65 are knitted in an inverted manner relative to the first portions of tricot stitches 64 (the direction of the lying V of the first portions is inverted relative to the direction of the lying V of the second portions), and in particular are offset relative to the first portions of tricot stitches 64 by two columns of stitches.

The first portions of tricot stitches 64 correspond to first knitted patterns M1 (<), a first pattern M1 extends for example between stitch (m1,n20), then stitch (m2,n18), and finally stitch (m3,n20).

The second portions of tricot stitches 65 correspond to second knitted patterns M2 (>), a second pattern M2 extends for example between stitch (m4,n16), then stitch (m5,n18), and finally stitch (m6,n16).

Advantageously, the second portions of tricot stitches 65 and the first portions of tricot stitches 64 are knitted by alternating in the warp direction or the height of the knitted fabric 20.

For example, a first portion of tricot stitches 64 extends over three columns of stitches n18 to n20 and over three rows of stitches m1 to m3; and a second portion of tricot stitches 65 extends over three columns of stitches n16 to n18 and over three rows of stitches m4 to m6. In particular, this second portion of tricot stitches 65 is offset by two columns of stitches, namely columns of stitches n19 and n20, relative to the first portion of tricot stitches 64.

Advantageously, the second knitted layer 70 comprises:
third portions 74 of tricot stitches each extending over three columns of stitches and over three rows of stitches, each in the shape of a lying V (“>”) open towards the first lateral edge 22, and
fourth portions 75 of tricot stitches each extending over three columns of stitches and over three rows of stitches, each in the shape of a lying V (“<”) open towards the second lateral edge 22,
the fourth portions of tricot stitches 75 are knitted in an inverted manner relative to the third portions of tricot stitches 74, and in particular are offset relative to the third portions of tricot stitches 74 by two columns of stitches.

The third portions of tricot stitches 74 correspond to third knitted patterns M3 (>), a third pattern M3 extends for example between stitch (m1,n18), then stitch (m2,n20), and finally stitch (m3,n18).

The fourth portions of tricot stitches 75 correspond to fourth knitted patterns M4 (<), a fourth pattern M4 extends for example between stitch (m4,n22), then stitch (m5,n20), and finally stitch (m6,n18).

Furthermore, the fourth portions of tricot stitches 75 and the third portions of tricot stitches 74 are knitted by being alternated in the warp direction or the height of the knitted fabric 20.

For example, a third portion of tricot stitches 74 extends over three columns of stitches n18 to n20 and over three rows of stitches m1 to m3; and a fourth portion of tricot stitches 75 extends over three columns of stitches n20 to n22 and over three rows of stitches m4 to m6. In particular, this fourth portion of tricot stitches 75 is offset by two columns of stitches, namely columns of stitches n18 and n19, relative to said third portion of tricot stitches 74.

Advantageously, the first knitted layer 60 is knitted in a direction opposite to the knitting direction of the second knitted layer 70, that is to say that the first guide bar supporting the yarn A 62 knits for example from left to right while the second guide bar supporting the yarn B 72 knits from right to left.

Advantageously, the third knitted layer 80 comprises:
fifth portions 84 of tricot stitches each extending over two columns of stitches and over three rows of stitches, each in the shape of a lying V (“>”) open towards the first lateral edge 22, and
sixth portions 85 of tricot stitches each extending over two columns of stitches and over three rows of stitches, each in the shape of a lying V (“<”) open towards the second lateral edge 24,
the sixth portions of tricot stitches 85 are knitted in an inverted manner relative to the fifth portions of tricot stitches 84 (the direction of the lying V of the fifth portions is inverted relative to the direction of the lying V of the sixth portions), and in particular are offset relative to the fifth portions of tricot stitches 84 by two columns of stitches.

Furthermore, the sixth portions of tricot stitches 85 and the fifth portions of tricot stitches 84 are knitted by being alternated in the warp direction or the height of the knitted fabric 20.

The fifth portions of tricot stitches 84 correspond to fifth knitted patterns M5 (>) open towards the first lateral edge 22, a fifth pattern M5 extends for example between stitch (m1,n2), then stitch (m2,n3), and finally stitch (m3,n2).

The sixth portions of tricot stitches 85 correspond to sixth knitted patterns M6 (<), a sixth pattern M6 extends for example between stitch (m4,n6), then stitch (m5,n5), and finally stitch (m6,n6).

For example, a fifth portion of tricot stitches 84 extends over two columns of stitches n2 to n3 and over three rows of stitches m1 to m3; and a sixth portion of tricot stitches 85 extends over two columns of stitches n5 to n6 and over three rows of stitches m4 to m6.

In particular, this sixth portion of tricot stitches 85 is offset by two columns of stitches, namely columns of stitches n3 and n4, relative to the fifth portion of tricot stitches 84.

Advantageously, the arrangement of the first, second, and third knitted layers (60, 70, 80) allows to arrange knitted sections around through openings 90.

In particular, the warp-knitted fabric 20 comprises a first knitted section 100 comprising tricot stitches, said first section 100 extends over three columns of stitches and over three rows of stitches, and is knitted with yarn A 62, yarn B 72, and yarn C 82. The warp-knitted fabric comprises a second knitted section 120 comprising tricot stitches, said second section extends over three columns of stitches and over three rows of stitches, and is knitted with yarn A 62, yarn B 72, and yarn C 82. The second knitted section 120 is spaced from the first knitted section 100 by a through orifice 90 having at least one dimension greater than or equal to 3 mm.

In particular, the first knitted section 100 is in knitted connection with the second knitted section 120 via a third knitted section 140, said third knitted section 140 comprising tricot stitches, said third section extends over three columns of stitches and over three rows of stitches, and is knitted with yarn A 62, yarn B 72, and yarn C 82.

In particular, the second knitted section 120 is in knitted connection with the first knitted section 100 also via a fourth knitted section 160, said fourth knitted section 160 comprising tricot stitches, said fourth section extends over three columns and three rows of stitches, and is knitted with yarn A 62, yarn B 72, and yarn C 82.

The first, second, third and fourth knitted sections (100, 120, 140, 160) are advantageously arranged in a circle so as to delimit a through opening 90.

Advantageously, each knitted section (100, 120, 140, 160) comprises a gripping inclined loop formed by a portion of the monofilament yarn C 82 of the third knitted layer 80.

The gripping loops 40A inclined towards the first lateral edge 22 are formed by the sixth lying V knitted patterns (<), and the gripping loops 40B inclined towards the second lateral edge 24 are formed by the fifth lying V (>) knitted patterns.

Advantageously, the gripping inclined loops 40 (40A, 40B) knitted with the monofilament yarn C 82 are each arranged between a first base stitch, for example the stitch 82a, and a second base stitch, for example the stitch 82b. In addition, each gripping inclined loop further comprises a first blocking stitch, for example the stitch 72a, knitted with the yarn B 70 and superimposed with the first base stitch, and a second blocking stitch, for example the stitch 72b, knitted with the yarn B 70 and superimposed with the second base stitch. These first and second blocking stitches are superimposed with first and second base stitches respectively and allow to hold the gripping loops firmly to the knitted bottom 30 without risk of the loops relaxing in length. The feet of a gripping loop being formed by the base and blocking stitches are thus blocked with the knitted bottom 30.

This arrangement thus allows to preserve the shape of the loops and therefore to improve their gripping power.

A gripping loop 40A extending between the first and second base stitches is referenced in FIG. 3. After knitting, the center stitch 82c unravels, that is to say, it is released from its needle, and forms the gripping loop 40A. Similarly, a gripping loop 40B comprises a center stitch 82c that unravels once the knitted fabric 20 is relaxed.

Advantageously, a gripping inclined loop, for example a gripping inclined loop 82c, is knitted on a row of stitches n and a column of stitches p, for example the column of stitches n9 and the row of stitches m5, and the knitted yarns A (62) and B (72) are knitted so as to cross on either side of the gripping knitted loop: on said column of stitches p, for example n9, and on a row of stitches n+1, for example m6, adjacent to the row of stitches n (for example m5), and on said row of stitches p, for example n9, and on a row of stitches n−1, for example m4, adjacent to the row of stitches n, p and n being whole numbers greater than or equal to 1.

In a specific preferred example, the knitting diagram:
of the first guide bar supporting said monofilament yarn A 62 for the formation of the first layer 60 is: 5-4/5-6/5-4/8-9/8-7/8-9//with a yarning: . . . I;
of the second guide bar supporting said monofilament yarn B 72 for the formation of the first layer 70 is: 1-0/2-3/1-0/4-5/3-2/4-5//with a yarning: . . . I;
of the third guide bar supporting said monofilament yarn C 82 for the formation of the first layer 70 is: 4-5/3-2/4-5/1-0/2-3/1-0//with a yarning: .I..

Monofilament yarn A 62 is a polypropylene monofilament yarn with a diameter of 0.10 mm. Monofilament yarn B 72 is a polypropylene monofilament yarn with a diameter of 0.10 mm. Monofilament yarn C 82 is a poly-L-lactic acid (PLLA) monofilament yarn with a diameter of 0.15 mm. In this specific example, the mass fraction of PLLA monofilament yarns, and therefore PLLA, in the warp-knitted fabric 20 is approximately 66%, and the mass fraction of PP monofilament yarns, and therefore PP, in the warp-knitted fabric is approximately 34%. The surface mass of the warp-knitted fabric 20 is approximately 105 g/m² at +/−10 g/m², the number of stitches in the warp direction per cm is approximately 3, and the number of stitches in the weft direction per cm is approximately 3.

In this specific example, the thickness E of the warp-knitted fabric, including the 40 gripping loops, is approximately 1.5 mm to within +/−0.5 mm. The breaking strength in the warp direction is greater than 80N, the elongation in the warp direction under a load of 80N is 110% maximum, the strength in the weft direction is greater than 80N, and the elongation in the weft direction under a load of 80N is 90% maximum. The mechanical tests are carried out in this specific example according to ISO 13934-1 and Annex 1, with the following parameters: 5 kN sensor, fabric dedicated jaw, tightening with the 4.5 Nm torque wrench, initial length of the sample 200 mm and width of approximately 50 mm, speed of 100 mm/min.

Monofilament yarn C 82 can advantageously be replaced by a monofilament yarn of substantially the same diameter, also bioresorbable, but in a copolymer of trimethylene carbonate (PTMC) and L-lactic acid.

The warp-knitted fabric 20 obtained comprises gripping and inclined loops formed in a regular and reproducible manner by knitting without it being necessary to merge portions of the knitted fabric after knitting. The loops thus formed allow immediate adhesion in the adipose tissues, allow the knitted fabric to be repositionable while maintaining satisfactory immediate adhesion. The arrangement of the gripping and inclined loops also allows to block migration of the warp-knitted fabric in the weft direction and the warp direction once placed against the adipose tissues. In addition, it is possible to roll the warp-knitted fabric on itself to arrange it in a knitted fabric without the loops becoming snagged in the structure of the knit, preventing its proper unwinding thereafter, and risking damaging it.

Finally, the gripping and inclined loops 40 are formed by portions of bioresorbable monofilament yarns whose feet are blocked in the mesh structure so that they are firmly anchored and non-deformable. In addition, the loops will resorb over time, and only the knitted bottom will remain in place to ensure stable mechanical reinforcement.

It is also possible to provide that yarns A and B are bioresorbable but in bioresorbable materials that are resorbed less quickly than the material forming yarn C of the gripping loops depending on the pathology to be treated, the compromise to be found between immediate and repositionable adhesion, and finally the duration sought for the mechanical reinforcement.

1. An implantable device for parietal surgery wherein said implantable device comprises a warp-knitted fabric, said knitted fabric comprising:
a first knitted layer comprising a monofilament yarn A knitted with tricot stitches,
a second knitted layer comprising a monofilament yarn B knitted with tricot stitches,
a third layer knitted with a monofilament yarn C knitted with tricot stitches, and
said warp-knitted fabric comprises a knitted bottom and comprises gripping knitted loops projecting from said knitted bottom and inclined towards said knitted bottom.

2. The implantable device according to claim 1, wherein the warp-knitted fabric comprises a first lateral edge and a second lateral edge, said first and second lateral edges extending in the warp direction of the warp-knitted fabric, and wherein said warp-knitted fabric comprises at least one set comprising first gripping loops oriented towards the first lateral edge and second gripping loops oriented towards the second lateral edge, said first gripping loops and said second gripping loops of said set extend alternately in the warp direction.

3. The implantable device according to claim 2, wherein the warp-knitted fabric comprises several sets of gripping and oriented loops, said sets being arranged between the first and second lateral edges.

4. The implantable device according to claim 1, wherein the first knitted layer comprises first portions of tricot stitches each extending over at least three columns of stitches, and second portions of tricot stitches each extending over at least three columns of stitches, the first portions of tricot stitches are alternated with the second portions of tricot stitches, and the second portions of tricot stitches are knitted in an inverted manner with respect to the first portions of tricot stitches.

5. The implantable device according to claim 1, wherein the warp-knitted fabric comprises:
a first knitted section comprising tricot stitches, said first knitted section extends over at least three columns of stitches and is knitted with at least yarns A and B, and yarn C, and
a second knitted section comprising tricot stitches, said second knitted section extends over at least three columns of stitches and is knitted with at least yarns A and B, and yarn C, and
the second knitted section is spaced from the first knitted section by a through orifice.

6. The implantable device according to claim 5, wherein the first knitted section is in knitted connection with the second knitted section via a third knitted section, said third knitted section comprising tricot stitches, said third knitted section extends over at least three columns of stitches and is knitted with at least yarns A and B, and yarn C.

7. The implantable device according to claim 5, wherein at least one of the first and second knitted sections comprises an inclined gripping loop, said inclined gripping loop comprising a portion of a monofilament yarn C.

8. The implantable device according to claim 1, characterized in that wherein at least one loop selected from said gripping inclined loops is knitted with the monofilament yarn C, and in that wherein said gripping inclined loop is arranged between a first base stitch and a second base stitch knitted with the monofilament yarn C, and in that wherein the warp-knitted fabric comprises a first blocking stitch knitted with yarn A or B and superimposed with the first base stitch, and a second blocking stitch knitted with yarn A or B and superimposed with the second base stitch.

9. The implantable device according to claim 1, wherein at least one loop selected from said gripping inclined loops is knitted with the monofilament yarn C, and said gripping inclined loop is knitted on a row of stitches n and a column of stitches p, and the knitted yarns A and B are knitted so as to cross on either side of the gripping knitted loop: on said column of stitches p and on a row of stitches n+1 adjacent to the row of stitches n, and on said column of stitches p and on a row of stitches n−1 adjacent to the row of stitches n, p and n being whole numbers greater than or equal to 1.

10. The implantable device according to claim 1, wherein the knitting diagram of the first layer is: 5-4/5-6/5-4/8-9/8-7/8-9//.

11. The implantable device according to claim 1, wherein the knitting diagram of the second layer is: 1-0/2-3/1-0/4-5/3-2/4-5//.

12. The implantable device according to claim 1, wherein the knitting diagram of the third layer (80) is: 4-5/3-2/4-5/1-0/2-3/1-0//.

13. The implantable device according to claim 1, wherein the warp-knitted fabric comprises at least 55% by mass of one or more bioresorbable (co)polymer(s).

14. The implantable device according to claim 1, wherein the warp-knitted fabric comprises less than 45% by mass of one or more non-resorbable (co)polymer(s) that is/are selected from: (co)polymers of at least one olefin, polyesters such as polyethylene terephthalate and polybutylene terephthalate.

15. The implantable device according to claim 1, wherein the warp-knitted fabric has a surface mass greater than or equal to 50 g/m² and less than or equal to 150 g/m².

16. The implantable device according to claim 1, wherein at least one of the yarns selected among the yarns A, B and C, has a diameter greater than or equal to 0.02 mm and less than or equal to 0.30 mm.

17. The implantable device according to claim 1, wherein the warp-knitted fabric comprises a number of stitches in the warp direction less than or equal to 6 per cm, and a number of stitches in the weft direction less than or equal to 6 per cm.

18. A method for manufacturing an implantable device for parietal surgery, wherein said method comprises a step of knitting a warp-knitted fabric on a knitting machine comprising:
knitting a first knitted layer with a first guide bar supporting at least one monofilament yarn A and forming tricot stitches,
knitting a second knitted layer with a second guide bar supporting at least one monofilament yarn B and forming tricot stitches,
knitting a third knitted layer with a third guide bar supporting at least one monofilament yarn C and forming tricot stitches, and
in that the movement of said first, second, and third guide bars is determined so as to obtain a warp-knitted fabric comprising a knitted bottom and comprising gripping knitted loops projecting from said knitted bottom and inclined towards said knitted bottom.

19. The manufacturing method according to claim 19, wherein at least one loop, selected from said gripping inclined loops, is knitted with the monofilament yarn C, and wherein said loop is arranged between a first base stitch and a second base stitch, and wherein the warp-knitted fabric comprises a first blocking stitch knitted with yarn A or B and superimposed with the first base stitch, and optionally a second blocking stitch knitted with yarn A or B and superimposed with the second base stitch.

20. The implantable device according to claim 13, wherein the one or more bioresorbable (co)polymer(s) is/are selected from: polylactic acid (PLA), polyglycolic acid (PGA), oxidized cellulose, polycaprolactone (PCL), polydioxanone (PDO), polytrimethylenecarbonate (TMC), a copolymer of trimethylene carbonate (PTMC) and L-lactic acid, polyvinyl alcohol (PVA), polyhydroxyalkanoate (PHA), Poly4-Hydroxybutyrate (P4HB), P4HB and P3HB block copolymers, a mixture thereof.
